(19) United States
(12) Patent Application Publication
(10) Pub. No.: US 2025/0256070 A1
(43) Pub. Date: Aug. 14, 2025

(54) CROSSING GUIDEWIRE WITH DEFLECTABLE TIP FOR ADVANCING THROUGH AN AORTIC VALVE

(71) Applicant: BOSTON SCIENTIFIC SCIMED, INC., Maple Grove, MN (US)

(72) Inventors: Chris Cullen, Galway (IE); Tim O'Connor, Galway (IE); John Lardner, Galway (IE); Gavin O'Brien, Dublin (IE)

(73) Assignee: BOSTON SCIENTIFIC SCIMED, INC., MAPLE GROVE, MN (US)

(21) Appl. No.: 19/051,745

(22) Filed: Feb. 12, 2025

Related U.S. Application Data
(60) Provisional application No. 63/552,909, filed on Feb. 13, 2024.

(51) Int. Cl. A61M 25/09; A61M 25/01

(52) U.S. Cl. CPC A61M 25/09; A61M 25/0138; A61M 2025/09108; A61M 2025/09175; A61M 2210/127

(57) ABSTRACT

A crossing guidewire that is adapted for use in crossing an aortic valve may include a proximal shaft segment, a distal shaft segment including a distal end, and a hinge point disposed between the proximal shaft segment and the distal shaft segment. The hinge point may be adapted to allow the distal shaft segment to pivot relative to the proximal shaft segment when the distal end of the distal shaft segment contacts the aortic valve.

### CROSS REFERENCE TO RELATED APPLICATIONS

This application claims the benefit of priority of U.S. Provisional Application No. 63/552,909 filed Feb. 13, 2024, the entire disclosure of which is hereby incorporated by reference.

### TECHNICAL FIELD

The present disclosure pertains to medical devices and methods for using medical devices. More particularly, the present disclosure pertains to a guidewire for accessing and advancing through an aortic valve.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a crossing guidewire that is adapted for use in crossing an aortic valve. The crossing guidewire includes an elongate shaft and a hinge point that divides the elongate shaft into a main segment and a pivoting segment. The hinge point is adapted to allow the pivoting segment to pivot relative to the main segment when the pivoting segment contacts the aortic valve, thereby centering the hinge point relative to the aortic valve.

Alternatively or additionally, the hinge point may be adapted to allow the pivoting segment to pivot more than 45 degrees relative to the main segment.

Alternatively or additionally, the hinge point may be adapted to allow the pivoting segment to pivot more than 60 degrees relative to the main segment.

Alternatively or additionally, the hinge point may be adapted to allow the pivoting segment to pivot more than 90 degrees relative to the main segment.

Alternatively or additionally, the hinge point may function as a new distal tip when subsequently advancing the crossing guidewire through the aortic valve after the pivoting segment has pivoted relative to the main segment.

Alternatively or additionally, the hinge point may include a ball and socket, where one of the main segment and the pivoting segment has a terminus forming a socket and the other of the main segment and the pivoting segment has a terminus forming a ball that rotatably fits within the socket.

Alternatively or additionally, the hinge point may include a gap between the proximal shaft segment and the distal shaft segment, with a flexible polymeric sleeve extending over the gap.

Alternatively or additionally, the hinge point may include a kink point that will preferentially bend when the distal end of the pivoting segment contacts the aortic valve.

Another example may be found in a crossing guidewire that is adapted for use in crossing an aortic valve. The crossing guidewire includes a proximal shaft segment, a distal shaft segment that includes a distal end, and a hinge point that is disposed between the proximal shaft segment and the distal shaft segment. The hinge point is adapted to allow the distal shaft segment to pivot relative to the proximal shaft segment when the distal end of the distal shaft segment contacts the aortic valve.

Alternatively or additionally, the distal shaft segment may have a length extending between the hinge point and the distal end that is sufficient to position the hinge point at or near a midpoint of the aortic valve when the distal shaft segment pivots relative to the proximal shaft segment.

Alternatively or additionally, the distal shaft segment may have a length extending between the hinge point and the distal end that is in a range of 10 millimeters to 15 millimeters.

Alternatively or additionally, the distal shaft segment may have a length extending between the hinge point and the distal end that is about 13 millimeters.

Alternatively or additionally, the hinge point may include a ball and socket, where one of the proximal shaft segment and the distal shaft segment has a terminus forming a socket and the other of the proximal shaft segment and the distal shaft segment has a terminus forming a ball that rotatably fits within the socket.

Alternatively or additionally, the hinge point may include a gap between the proximal shaft segment and the distal shaft segment, with a flexible polymeric sleeve extending over the gap.

Alternatively or additionally, the hinge point may include a kink point that will preferentially bend when the distal end of the distal shaft segment bottoms out within the aortic valve.

Alternatively or additionally, the distal end of the distal shaft segment may include a rounded tip.

Another example may be found in a method for accessing the left ventricle through the aortic valve. The method includes advancing a first catheter through the aortic arch, the first catheter positioned with a distal end of the first catheter proximate the sinotubular junction. A crossing guidewire is advanced through the first catheter and towards the aortic valve, the crossing guidewire including an elongate shaft and a hinge point dividing the elongate shaft into a main segment and a pivoting segment. The crossing guidewire is urged distally until a distal end of the pivoting segment contacts one of the cusps of the aortic valve. The crossing guidewire is urged further distally, thereby causing the pivoting segment to pivot relative to the main segment and thereby causing the hinge point of the crossing wire to be more closely centered relative to the aortic valve. The crossing guidewire is urged further distally, thereby causing the hinge point of the crossing wire to extend through the aortic valve.

Alternatively or additionally, causing the pivoting segment to pivot relative to the main segment may include causing the pivoting segment to pivot at least 45 degrees relative to the main segment.

Alternatively or additionally, causing the pivoting segment to pivot relative to the main segment may include causing the pivoting segment to pivot at least 60 degrees relative to the main segment.

Alternatively or additionally, causing the pivoting segment to pivot relative to the main segment may include causing the pivoting segment to pivot at least 90 degrees relative to the main segment.

The above summary of some embodiments, aspects, and/or examples is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of a human heart;

FIG. 2 is a schematic view of an aorta;

FIG. 3 is a schematic superior view of the aorta of FIG. 2, including an aortic valve;

FIGS. 4A through 4H are schematic views of the aorta, showing an illustrative method for reaching and passing through the aortic valve;

FIG. 5 is a schematic view of an illustrative crossing wire that may be utilized in the illustrative method shown in FIGS. 4A through 4H;

FIG. 6A is a cross-sectional view taken along line 6-6 of FIG. 5, with the illustrative crossing wire shown in a linear configuration;

FIG. 6B is a cross-sectional view taken along line 6-6 of FIG. 5, with the illustrative crossing wire shown in a deflected configuration;

FIG. 7 is a schematic view of an illustrative crossing wire that may be utilized in the illustrative method shown in FIGS. 4A through 4H;

FIG. 8A is a cross-sectional view taken along line 8-8 of FIG. 7, with the illustrative crossing wire shown in a linear configuration;

FIG. 8B is a cross-sectional view taken along line 8-8 of FIG. 7, with the illustrative crossing wire shown in a deflected configuration;

FIG. 9A is a schematic view of an illustrative crossing wire that may be utilized in the illustrative method shown in FIGS. 4A through 4H, with the illustrative crossing wire shown in a linear configuration; and

FIG. 9B is a schematic view of the illustrative crossing wire of FIG. 9A, shown in a linear configuration.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications may be disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of a device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians. In some instances, these therapies may be referred to as transaortic valve implantation or transcatheter aortic valve implantation, also known as TAVI procedures. In some instances, these therapies may be referred to as transaortic valve replacement or transcatheter aortic valve replacement, also known as TAVR procedures. The terms TAVI and TAVR may in some cases be used interchangeably to refer to a procedure in which a native aortic valve is repaired or replaced. In some instances, a TAVI or TAVR procedure may be utilized to install a second replacement aortic valve in place of a previously installed first replacement aortic valve.

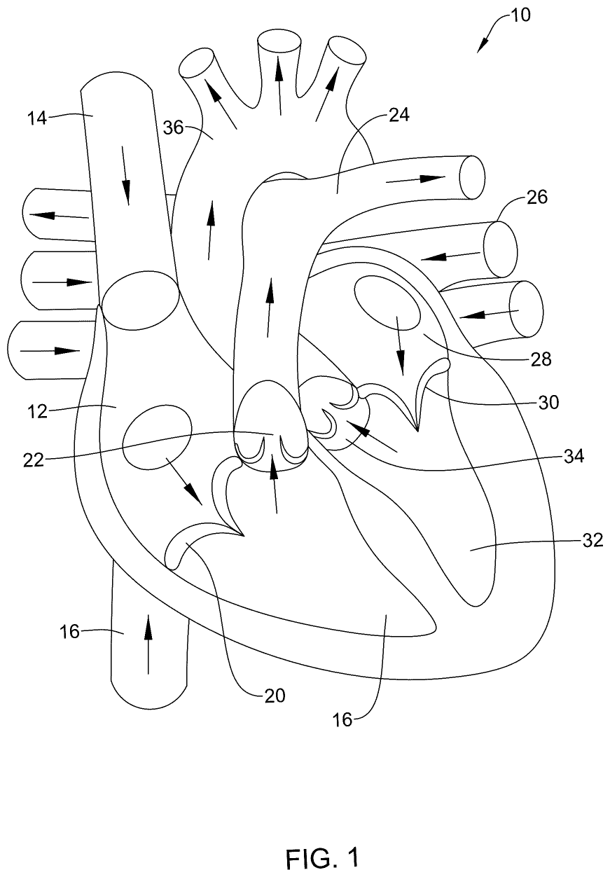

FIG. 1 is a schematic view of a human heart 10, illustrating some of the anatomy involved in accessing the interior of the heart 10 in general and in accessing the aortic valve in particular. The heart 10 includes a right atrium 12, which receives blood via the superior vena cava 14 and the inferior vena cava 16. Blood within the right atrium 12 passes to a right ventricle 18 via a tricuspid valve 20. Blood within the right ventricle 18 passes through a pulmonary valve 22 and into a pulmonary artery 24 in order to go to the lungs to be oxygenated. Oxygenated blood from the lungs returns via pulmonary veins 26 and enters a left atrium 28. A mitral valve 30 allows blood to flow from the left atrium 28 into a left ventricle 32. Blood exits the left ventricle 32, through an aortic valve 34, into an aorta 36 and from there is carried throughout the body.

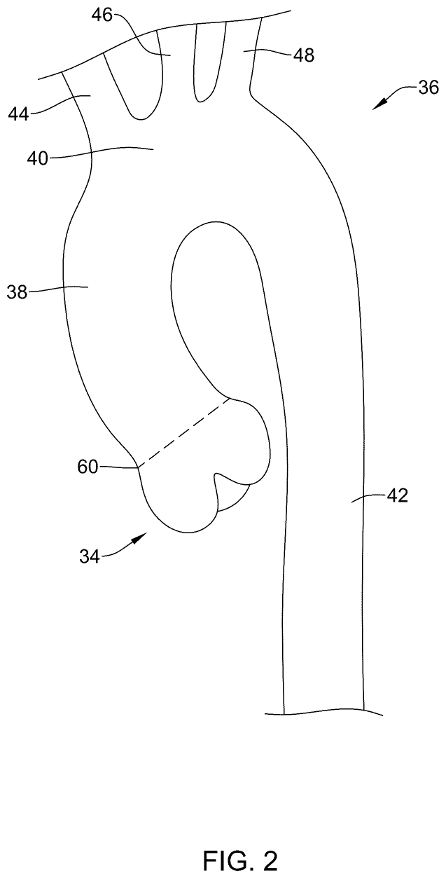

FIG. 2 is a schematic view of the aorta 36. As can be seen, the aorta 36 includes an ascending aorta 38 that extends from the aortic valve 34 up to an aortic arch 40, as well as a descending aorta 42 that extends downward from the aortic arch 40. Several arteries extend from the aortic arch 40. For example, a brachiocephalic artery 44 that bifurcates into a right subclavian artery and a right common carotid artery extends from the aortic arch 40. A left common carotid artery 46 and a left subclavian artery 48 also extend from the aortic arch 40. It will be appreciated that the aortic valve 34 may be reached by an intravascular device extending into the body via the brachiocephalic artery 44, the common carotid artery 46, the left subclavian artery 48 or a femoral artery (not shown). Access through the femoral artery follows a path up to the abdominal aorta (not shown), through the descending aorta 42, the aortic arch 40 and the ascending aorta 38. This path may be considered as moving upstream, against the normal flow of blood.

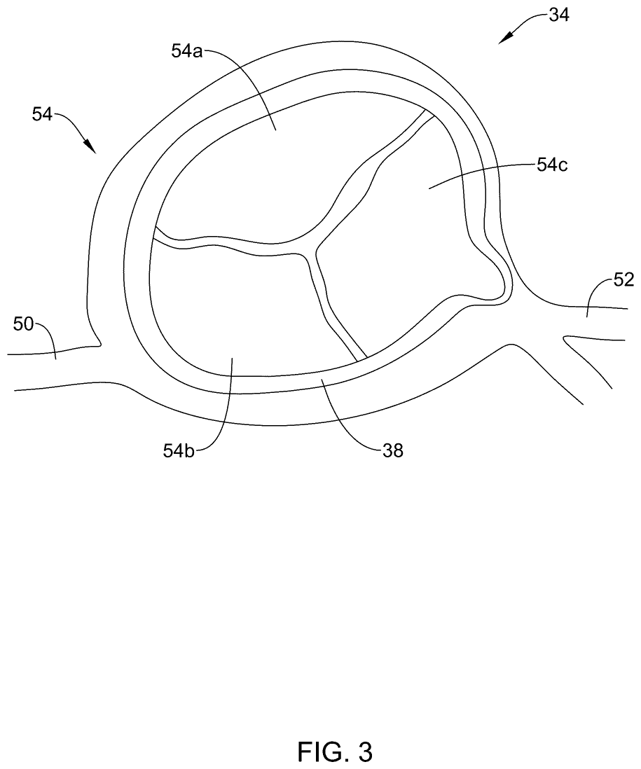

FIG. 3 is a schematic view of the aortic valve 34, looking down on the aortic valve 34 from within the ascending aorta 38. A small part of the ascending aorta 38 is shown, showing a portion of a right coronary artery 50 and a left main coronary artery 52. The aortic valve 34 includes a total of three valve cusps 54, individually labeled as 54a, 54b and 54c. Each of the valve cusps 54 may be considered as being bowl-shaped, particularly when closed (as shown). Each of the valve cusps 54 are attached along one side to the aorta 36 and have an opposing edge that seals against the adjoining valve cusps 54 when the aortic valve 34 is closed. It will be appreciated that trying to extend an intravascular device such as a guidewire through the aortic valve 34 may be problematic, as a distal end of the intravascular device may tend to bottom out in one of the bowl-shaped valve cusps 54, especially if the aortic valve 34 is closed at the time that the intravascular device is extended distally.

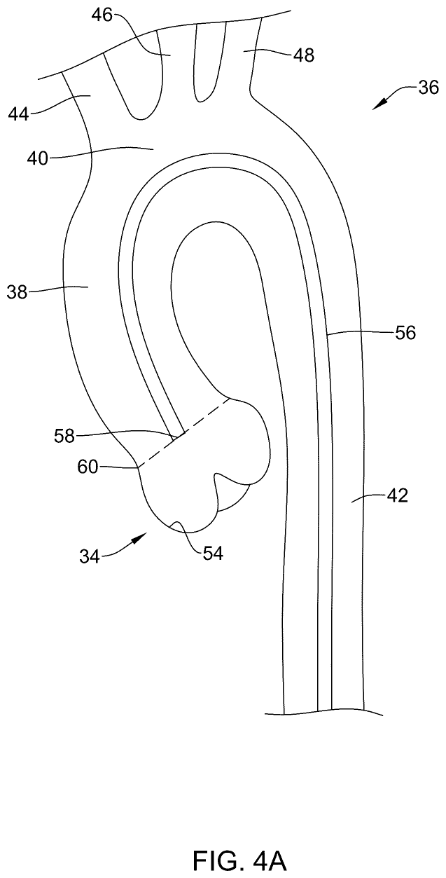

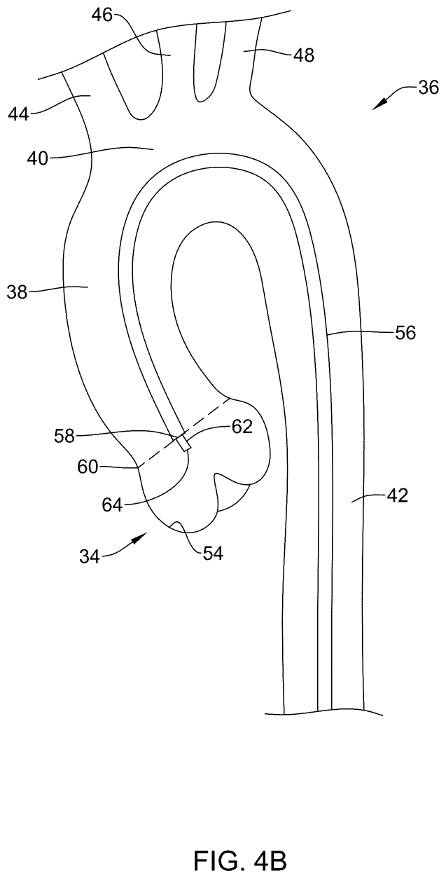

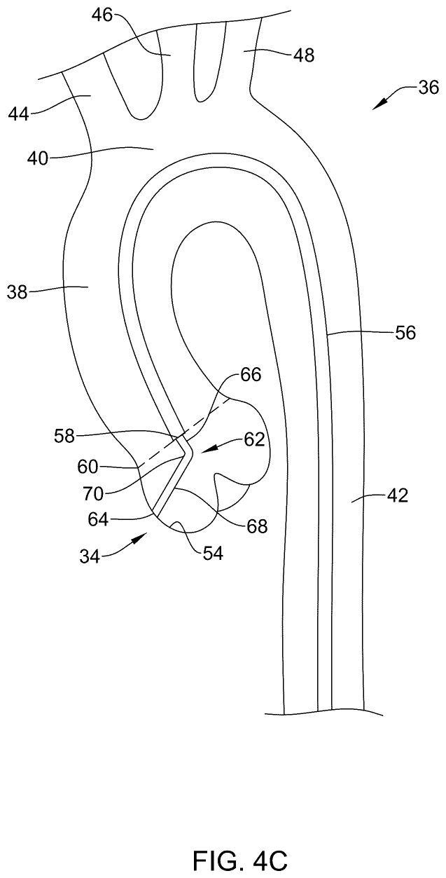

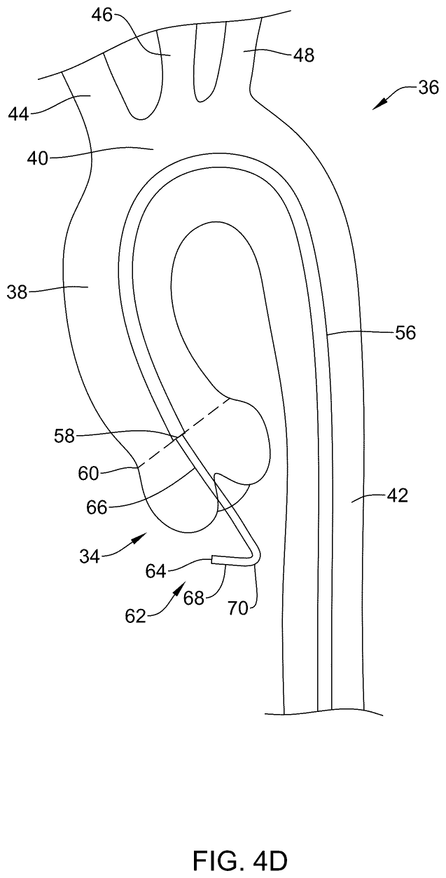

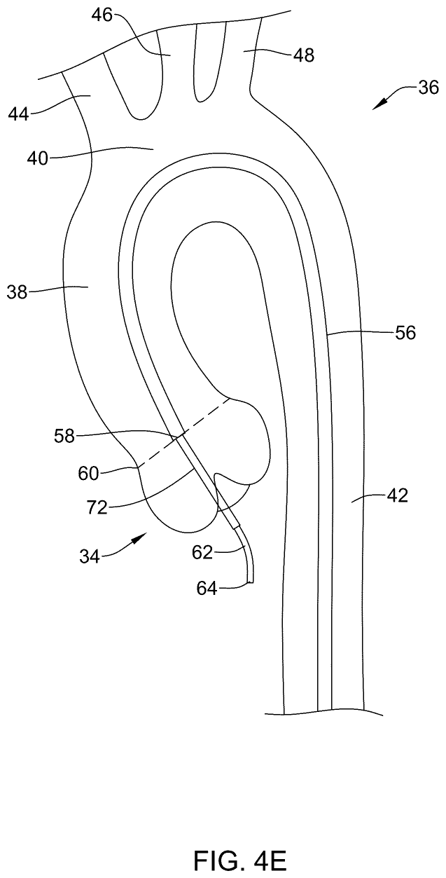

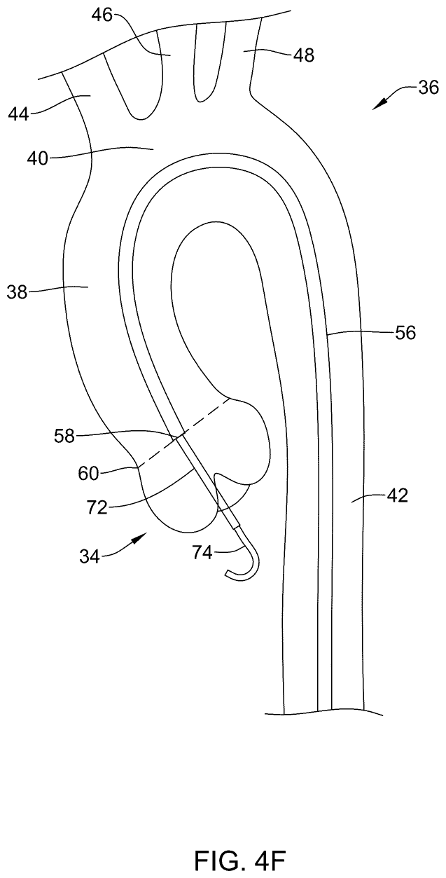

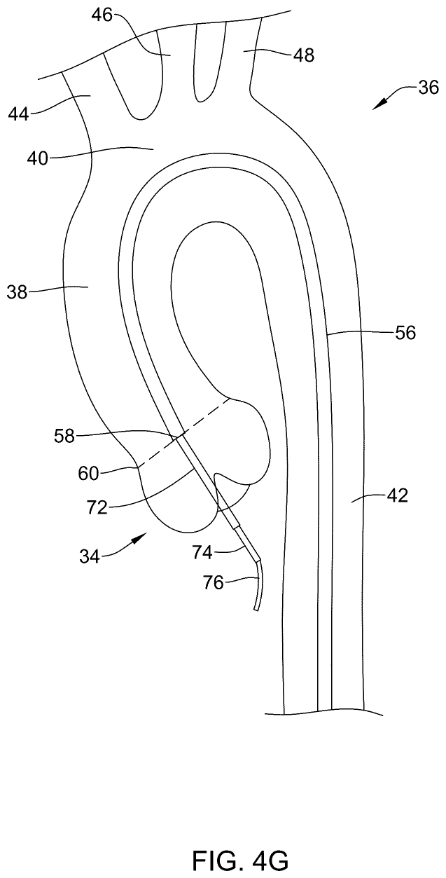

In some instances, a crossing guidewire may be adapted to facilitate crossing the aortic valve 34. In some instances, as will be discussed, a crossing guidewire may be adapted to have a distal end that bottoms out in, or otherwise contacts, one of the valve cusps 54, thereby causing a distal end region of the crossing guidewire to deflect or pivot. In some instances, this may cause the deflection point or pivot point, sometimes referred to as a hinge point, to be more closely centered relative to the aortic valve 34 such that the deflection point or pivot point functions as a new distal end of the crossing guidewire and thus can more easily be extended through the aortic valve 34. FIGS. 4A through 4H show an illustrative method in which a crossing guidewire that has been adapted to facilitate crossing the aortic valve 34 is used in combination with other intravascular devices to reach and extend through the aortic valve 34.

In FIG. 4A and FIG. 4B, a first catheter 56 is advanced through the aorta 36 until a distal end 58 of the first catheter 56 is positioned proximate the STJ (sinotubular junction) 60. The STJ 60 denotes the bottom of the ascending aorta 38. In some cases, the first catheter 56 may be considered a guide catheter. In some instances, the first catheter 56 may be considered as being an AL1 catheter. Once the first catheter 56 is positioned, a crossing guidewire 62 may be advanced within the first catheter 56 such that a distal end 64 just extends through the distal end 58 of the first catheter 56. In some cases, the distal end 64 includes a rounded or otherwise atraumatic tip.

As seen in FIG. 4C, the crossing guidewire 62 has been urged distally until the distal end 64 of the crossing guidewire 62 has contacted one of the valve cusps 54. As can be seen, the crossing guidewire 62 includes a proximal shaft segment 66, a distal shaft segment 68 and a hinge point 70 that is disposed between the proximal shaft segment 66 and the distal shaft segment 68. The hinge point 70 may take several forms, as will be discussed. In some instances, the hinge point 70 may be considered as being a point along the crossing guidewire 62 where the distal shaft segment 68 is preferentially designed to deflect or pivot relative to the proximal shaft segment 66. In some instances, the hinge point 70 allows better centering of the proximal shaft segment 66 once the distal shaft segment 68 bottoms out within, or otherwise contacts, one of the valve cusps 54.

In some cases, the proximal shaft segment 66 may be considered as being a main shaft segment and the distal shaft segment 68 may be considered as being a pivoting segment. In some instances, the proximal shaft segment 66 and the distal shaft segment 68 may together be considered as defining an elongate shaft. With the distal end 64 of the crossing guidewire 62 bottomed out within, or otherwise contacting, one of the valve cusps 54, it can be seen that the hinge point 70 is more closely centered relative to the aortic valve 34, and is effectively a new distal end of the crossing guidewire 62. As seen in FIG. 4D, the crossing wire 62 may be further urged distally through the aortic valve 34 with the distal shaft segment 68 still pivoted relative to the proximal shaft segment 66. In some cases, having the proximal shaft segment 66 helps to center the hinge point 70.

Once the crossing wire 62 has been advanced as shown in FIG. 4D, a second catheter 72 may be advanced over the crossing wire 62 to a point where the second catheter 72 extends through the aortic valve 34 as shown in FIG. 4E. In some cases, the second catheter 72 may be considered as being a guide sheath. In some cases, instead of advancing the second catheter 72 over the crossing guidewire 62, the first catheter 56 may instead be advanced distally over the crossing guidewire 62 and through the aortic valve 34. In some instances, advancing the second catheter 72 (or alternatively, advancing the first catheter 56) over the crossing guidewire 62 may cause the distal shaft segment 68 to become realigned axially with the proximal shaft segment 66, thereby making it easier to subsequently advance other intravascular devices over the crossing guidewire 62. In some cases, once the second catheter 72 (or the first catheter 56) has been advanced distally through the aortic valve 34, the crossing guidewire 62 may be withdrawn proximally.

As seen in FIG. 4F, a pigtail catheter 74 may be advanced through the first catheter 56 and/or the second catheter 72. In some cases, the pigtail catheter 74 may be advanced over the crossing guidewire 62. In some instances, the pigtail catheter 74 may be advanced within the first catheter 56 and/or the second catheter 72 after the crossing guidewire 62 has been withdrawn. The pigtail catheter 74 may be used to inject a contrast fluid, for example. As seen in FIG. 4G, a guidewire 76 may be advanced through the first catheter 56 and/or the second catheter 72 and through the pigtail catheter 74. The guidewire 76 may be adapted for use in performing particular steps of a TAVI/TAVR procedure, for example. One or more additional intravascular devices may be advanced over the guidewire 76, for example. After the guidewire 76 has been advanced through the aortic valve 34, the pigtail catheter 74 may subsequently be withdrawn, as shown in FIG. 4H.

The crossing guidewire 62 may take a variety of different forms, as long as the crossing guidewire 62 has the hinge point 70 that is adapted to allow the distal shaft segment 68 to deflect or pivot relative to the proximal shaft segment 66, thereby allowing the hinge point 70 to become more closely centered relative to the aortic valve 34 and thus more likely aligned to pass through the aortic valve 34 when the crossing guidewire 62 is advanced distally. In some instances, the distal shaft segment 68 has a length extending between the hinge point 70 and the distal end 64 that is sufficient to position the hinge point 70 at or near a midpoint of the aortic valve 34 when the distal shaft segment 68 pivots relative to the proximal shaft segment 66. In some cases, the distal shaft segment 68 may have a length extending between the hinge point 70 and the distal end 64 that is in a range of 10 millimeters to 15 millimeters. In some cases, the distal shaft segment 68 may have a length extending between the hinge point and the distal end that is about 13 millimeters.

In some instances, the hinge point 70 may be adapted to allow the distal shaft segment (or pivoting segment) 68 to pivot more than 45 degrees relative to the proximal shaft segment (or main segment) 66. In some instances, the hinge point 70 may be adapted to allow the distal shaft segment (or pivoting segment) 68 to pivot more than 60 degrees relative to the proximal shaft segment (or main segment) 66. In some instances, the hinge point 70 may be adapted to allow the distal shaft segment (or pivoting segment) 68 to pivot more than 90 degrees relative to the proximal shaft segment (or main segment) 66. In some cases, there may be a relationship between the length of the distal shaft segment 68 and the maximum angle that the hinge point 70 is capable of reaching. For example, a greater length for the distal shaft segment 68 may mean that the maximum angle for the hinge point 70 is less. As another example, a shorter length for the distal shaft segment 68 may mean that the maximum angle for the hinge point 70 is greater. Essentially, there may be a geometric relationship between the length of the distal shaft segment 68 and how far (to what angle) the distal shaft segment 68 is able to deflect.

FIG. 5 is a schematic view of an illustrative crossing guidewire 80 that may be considered as being an example of the crossing guidewire 62. The crossing guidewire 80 includes an elongate shaft 82 that is divided into a proximal shaft segment 84 and a distal shaft segment 86 by a hinge point 88 that is disposed between the proximal shaft segment 84 and the distal shaft segment 86. In some instances, the hinge point 88 may include a ball and socket arrangement in which one of the proximal shaft segment 84 and the distal shaft segment 86 has a terminus forming a socket and the other of the proximal shaft segment 84 and the distal shaft segment 86 has a terminus forming a ball that rotatably fits within the socket.

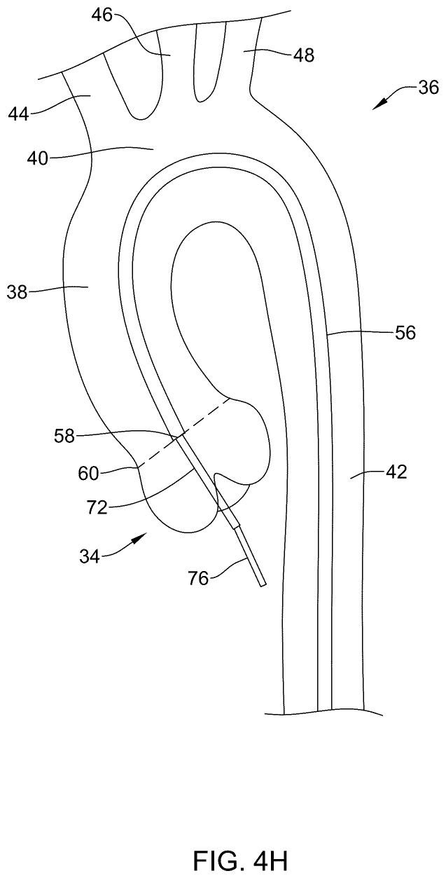

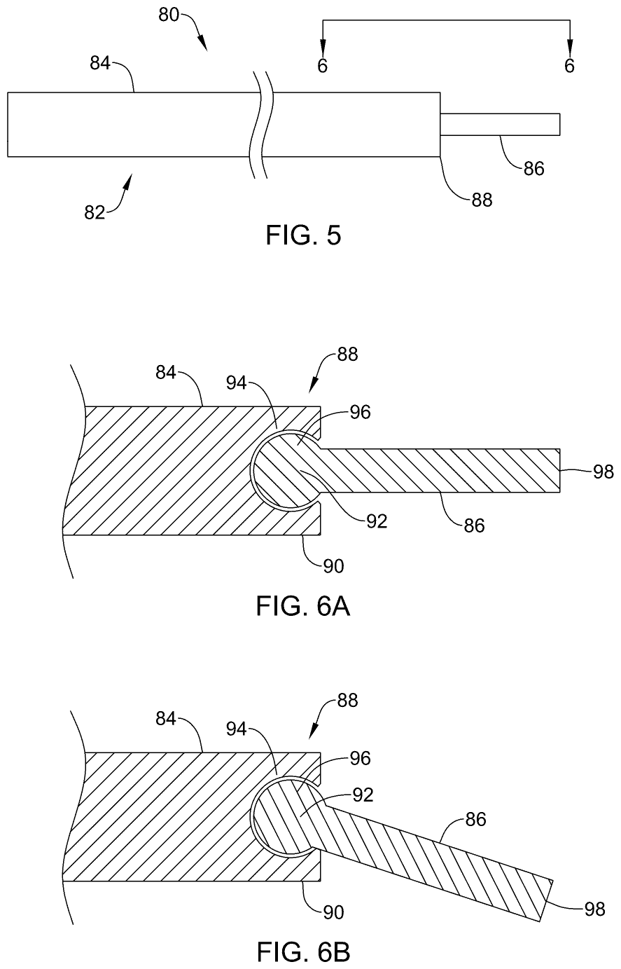

FIG. 6A is a cross-sectional view taken along the line 6-6 of FIG. 5. As shown, a terminus 90 of the proximal shaft segment 84 includes a rounded socket 94 and a terminus 92 of the distal shaft segment 86 includes a rounded ball portion 96 that fits within the rounded socket 94 and allows the distal shaft segment 86 to pivot within the rounded socket 94. In some cases, a frictional fit between the rounded ball portion 96 and the rounded socket 94 may help to hold the distal shaft segment 86 in a linear relationship with the proximal shaft segment 84, as shown in FIG. 6A. In FIG. 6B, which shows the crossing guidewire 80 in a deflected position, having a distal end 98 of the distal shaft segment 86 bottom out within one of the valve cusps 54 is sufficient to overcome any frictional forces between the rounded ball portion 96 and the rounded socket 94, thereby allowing the distal shaft segment 86 to deflect or pivot.

As shown, the distal shaft segment 86 has a reduced diameter relative to that of the proximal shaft segment 84. As an example, the distal shaft segment 86 may have a diameter that is reduced up to about 60 percent relative to a diameter of the proximal shaft segment 84. In some cases, the distal shaft segment 86 may have a diameter that is equal to that of the proximal shaft segment 84. In some instances, the distal shaft segment 86 has a length that is sufficient to position the hinge point 88 at or near a midpoint of the aortic valve 34 when the distal shaft segment 86 pivots relative to the proximal shaft segment 84. In some cases, the distal shaft segment 86 may have a length that is in a range of 10 millimeters to 15 millimeters. In some cases, the distal shaft segment 86 may have a length that is about 13 millimeters.

In some instances, the hinge point 88 may be adapted to allow the distal shaft segment (or pivoting segment) 86 to pivot more than 45 degrees relative to the proximal shaft segment (or main segment) 84. In some instances, the hinge point 88 may be adapted to allow the distal shaft segment (or pivoting segment) 86 to pivot more than 60 degrees relative to the proximal shaft segment (or main segment) 84. In some instances, the hinge point 88 may be adapted to allow the distal shaft segment (or pivoting segment) 86 to pivot more than 90 degrees relative to the proximal shaft segment (or main segment) 84. In some cases, there may be a geometric relationship between the length of the distal shaft segment 86 and how far (to what angle) the distal shaft segment 84 is able to deflect.

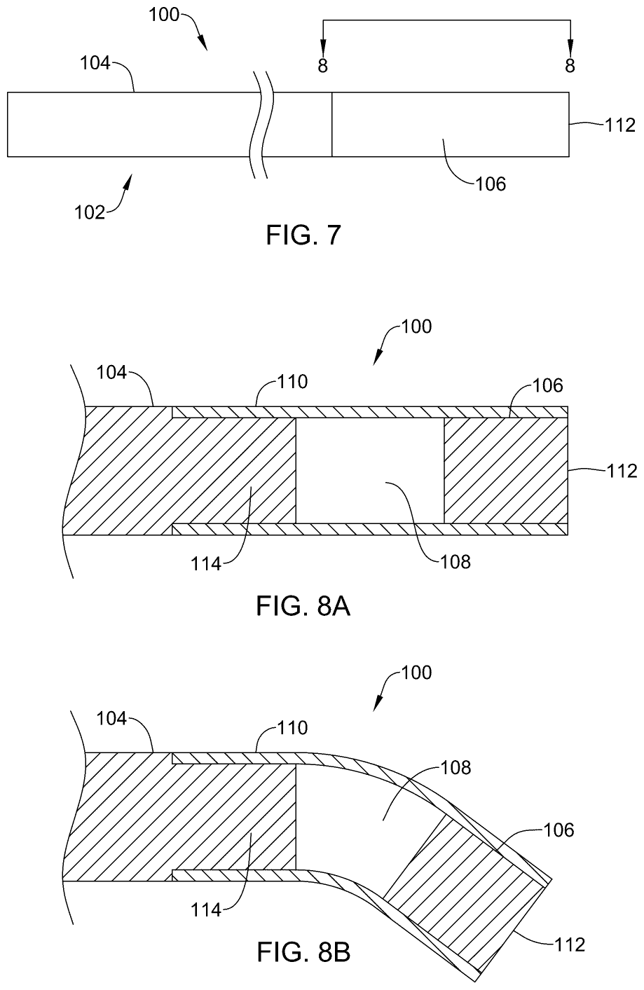

FIG. 7 is a schematic view of an illustrative crossing guidewire 100 that may be considered as being an example of the crossing guidewire 62. FIG. 8A is a cross-sectional view taken along the line 8-8 of FIG. 7, showing internal structure of the crossing guidewire 100. The crossing guidewire 100 includes an elongate shaft 102 that is divided into a proximal shaft segment 104 and a distal shaft segment 106 by a hinge point 108 that is disposed between the proximal shaft segment 104 and the distal shaft segment 106. In some cases, as shown, the hinge point 108 includes a physical gap between the proximal shaft segment 104 and the distal shaft segment 106. A flexible polymeric sleeve 110 extends over the gap to complete the hinge point 108.

In some instances, the flexible polymeric sleeve 110 may be formed of a heat-shrinkable material such as polyether block amides available commercially under the Pebax™ name, PET (polyethlene terephthalate), PTFE (polytetrafluoroethylene), PEF (polyethylene furanoate), PES (polyethersulfone), silicone rubber, high performance fluoroelastomers available commercially under the Viton™ name, polyolefins, PFA (perfluoroalkoxy) polymers, or PVDF (polyvinylidene fluoride). In some cases, the flexible polymeric sleeve 110 may be formed of a non-heat-shrinkable material that can form the hinge point 108. As an example, the flexible polymeric sleeve 110 may be formed of a silicone rubber having a Young's Modulus ranging from 0.448 MPa (mega Pascal) to 145 MPa. As another example, the flexible polymeric sleeve 110 may be formed of Pebax™ 7233, which has a Young's Modulus of 510 MPa.

Absent applied forces, the flexible polymeric sleeve 110 may hold the distal shaft segment 106 in a linear or axially aligned configuration (as shown in FIG. 8A). When a distal end 112 of the distal shaft segment 106 bottoms out within, or contacts, one of the valve cusps 54, the flexible polymeric sleeve 110 is flexible enough to allow the distal shaft segment 106 to deflect or pivot relative to the proximal shaft segment 104, as shown for example in FIG. 8B.

In some cases, the proximal shaft segment 104 may include a reduced diameter section 114 that accommodates the flexible polymeric sleeve 110 without increasing an overall diameter of the crossing guidewire 100. As an example, the proximal shaft segment 104 may have a maximum diameter of 0.965 millimeters (0.038 inches) and a minimum diameter (within the reduced diameter section 114) of 0.254 millimeters (0.010 inches). The distal shaft segment 106 may have a diameter of 0.010 inches. In some cases, the proximal shaft segment 104 may not include the reduced diameter section 114, in which case the flexible polymeric sleeve 110 may increase the overall diameter of the crossing guidewire 100.

In some instances, the distal shaft segment 106 has a length that is sufficient to position the hinge point 108 at or near a midpoint of the aortic valve 34 when the distal shaft segment 106 pivots relative to the proximal shaft segment 104. In some cases, the distal shaft segment 106 may have a length that is in a range of 10 millimeters to 15 millimeters. In some cases, the distal shaft segment 106 may have a length that is about 13 millimeters. In some instances, the hinge point 108 may be adapted to allow the distal shaft segment 106 to pivot more than 45 degrees relative to the proximal shaft segment 104. In some instances, the hinge point 108 may be adapted to allow the distal shaft segment 106 to pivot more than 60 degrees relative to the proximal shaft segment 104. In some instances, the hinge point 108 may be adapted to allow the distal shaft segment 106 to pivot more than 90 degrees relative to the proximal shaft segment 104. In some cases, there may be a geometric relationship between the length of the distal shaft segment 106 and how far (to what angle) the distal shaft segment 106 is able to deflect.

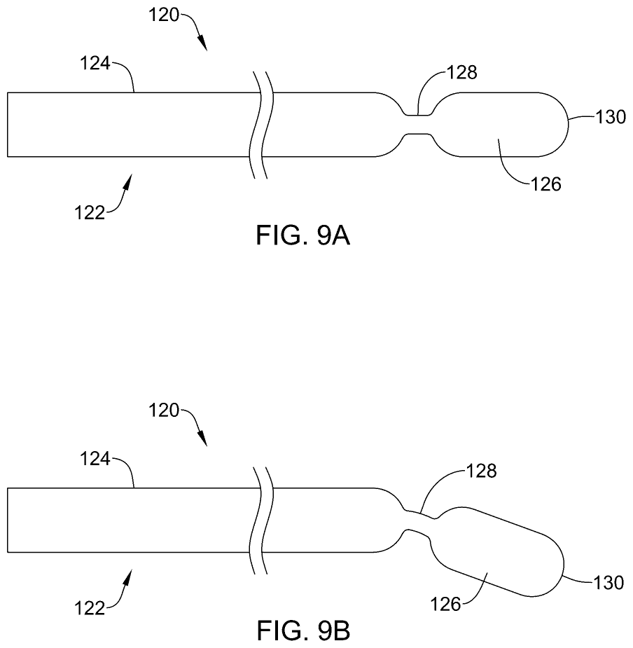

FIG. 9A is a schematic view of an illustrative crossing guidewire 120 that may be considered as being an example of the crossing guidewire 62. The crossing guidewire 120 includes an elongate shaft 122 that is divided into a proximal shaft segment 124 and a distal shaft segment 126 by a hinge point 128 that is disposed between the proximal shaft segment 124 and the distal shaft segment 126. In some cases, as shown, the hinge point 128 includes a reduced diameter segment or a kink point that will preferentially bend when a distal end 130 of the distal shaft segment 126 bottoms out within, or contacts, the aortic valve. Absent applied forces, the hinge point 128 may hold the distal shaft segment 126 in a linear or axially aligned configuration (as shown in FIG. 9A). When the distal end 130 of the distal shaft 126 bottoms out within, or contacts, one of the valve cusps 54, the hinge point 128 will preferentially bend in order to enough to allow the distal shaft segment 126 to deflect or pivot relative to the proximal shaft segment 124, as shown for example in FIG. 9B.

In some instances, the distal shaft segment 126 has a length that is sufficient to position the hinge point 128 at or near a midpoint of the aortic valve 34 when the distal shaft segment 126 pivots relative to the proximal shaft segment 124. In some cases, the distal shaft segment 126 may have a length that is in a range of 10 millimeters to 15 millimeters. In some cases, the distal shaft segment 126 may have a length that is about 13 millimeters. In some instances, the hinge point 128 may be adapted to allow the distal shaft segment 126 to pivot more than 45 degrees relative to the proximal shaft segment 124. In some instances, the hinge point 128 may be adapted to allow the distal shaft segment 126 to pivot more than 60 degrees relative to the proximal shaft segment 124. In some instances, the hinge point 128 may be adapted to allow the distal shaft segment 126 to pivot more than 90 degrees relative to the proximal shaft segment 124. In some cases, there may be a geometric relationship between the length of the distal shaft segment 126 and how far (to what angle) the distal shaft segment 126 is able to deflect.

The materials that can be used for the various components of the devices and components thereof disclosed herein may include those commonly associated with medical devices. In some instances, the devices and/or other elements disclosed herein may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some instances, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some instances, portions or all of the device and/or other elements disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the devices and/or other elements disclosed herein. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the devices and/or other elements disclosed herein to achieve the same result.

In some instances, a degree of Magnetic Resonance Imaging (MRI) compatibility is imparted into the devices and/or other elements disclosed herein. For example, the devices and/or components or portions thereof may be made of a material that does not substantially distort the image and create substantial artifacts (e.g., gaps in the image). Certain ferromagnetic materials, for example, may not be suitable because they may create artifacts in an MRI image. The medical device(s) or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some instances, the devices and/or other elements disclosed herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some instances the sheath can be blended with a liquid crystal polymer (LCP). For example, the mixture can contain up to about 6 percent LCP.

In some instances, the devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A crossing guidewire adapted for use in crossing an aortic valve, the crossing guidewire comprising:
an elongate shaft; and
a hinge point dividing the elongate shaft into a main segment and a pivoting segment;
wherein the hinge point is adapted to allow the pivoting segment to pivot relative to the main segment when the pivoting segment contacts the aortic valve, thereby centering the hinge point relative to the aortic valve.

2. The crossing guidewire of claim 1, wherein the hinge point is adapted to allow the pivoting segment to pivot more than 45 degrees relative to the main segment.

3. The crossing guidewire of claim 1, wherein the hinge point is adapted to allow the pivoting segment to pivot more than 60 degrees relative to the main segment.

4. The crossing guidewire of claim 1, wherein the hinge point is adapted to allow the pivoting segment to pivot more than 90 degrees relative to the main segment.

5. The crossing guidewire of claim 1, wherein the hinge point functions as a new distal tip when subsequently advancing the crossing guidewire through the aortic valve after the pivoting segment has pivoted relative to the main segment.

6. The crossing guidewire of claim 1, wherein the hinge point comprises a ball and socket, where one of the main segment and the pivoting segment has a terminus forming a socket and the other of the main segment and the pivoting segment has a terminus forming a ball that rotatably fits within the socket.

7. The crossing guidewire of claim 1, wherein the hinge point comprises a gap between the proximal shaft segment and the distal shaft segment, with a flexible polymeric sleeve extending over the gap.

8. The crossing guidewire of claim 1, wherein the hinge point comprises a kink point that will preferentially bend when the distal end of the pivoting segment contacts the aortic valve.

9. A crossing guidewire adapted for use in crossing an aortic valve, the crossing guidewire comprising:
a proximal shaft segment;
a distal shaft segment including a distal end; and
a hinge point disposed between the proximal shaft segment and the distal shaft segment, the hinge point adapted to allow the distal shaft segment to pivot relative to the proximal shaft segment when the distal end of the distal shaft segment contacts the aortic valve.

10. The crossing guidewire of claim 9, wherein the distal shaft segment has a length extending between the hinge point and the distal end that is sufficient to position the hinge point at or near a midpoint of the aortic valve when the distal shaft segment pivots relative to the proximal shaft segment.

11. The crossing guidewire of claim 9, wherein the distal shaft segment has a length extending between the hinge point and the distal end that is in a range of 10 millimeters to 15 millimeters.

12. The crossing guidewire of claim 9, wherein the distal shaft segment has a length extending between the hinge point and the distal end that is about 13 millimeters.

13. The crossing guidewire of claim 9, wherein the hinge point comprises a ball and socket, where one of the proximal shaft segment and the distal shaft segment has a terminus forming a socket and the other of the proximal shaft segment and the distal shaft segment has a terminus forming a ball that rotatably fits within the socket.

14. The crossing guidewire of claim 9, wherein the hinge point comprises a gap between the proximal shaft segment and the distal shaft segment, with a flexible polymeric sleeve extending over the gap.

15. The crossing guidewire of claim 9, wherein the hinge point comprises a kink point that will preferentially bend when the distal end of the distal shaft segment contacts the aortic valve.

16. The crossing guidewire of claim 9, wherein the distal end of the distal shaft segment comprises a rounded tip.

17. A method for accessing the left ventricle through the aortic valve, the method comprising:
advancing a first catheter through the aortic arch, the first catheter positioned with a distal end of the first catheter proximate the sinotubular junction;
advancing a crossing guidewire through the first catheter and towards the aortic valve, the crossing guidewire including an elongate shaft and a hinge point dividing the elongate shaft into a main segment and a pivoting segment;
urging the crossing guidewire distally until a distal end of the pivoting segment contacts one of the cusps of the aortic valve;
further urging the crossing guidewire distally, thereby causing the pivoting segment to pivot relative to the main segment and thereby causing the hinge point of the crossing wire to be more closely centered relative to the aortic valve; and
further urging the crossing wire distally, thereby causing the hinge point of the crossing wire to extend through the aortic valve.

18. The method of claim 17, wherein causing the pivoting segment to pivot relative to the main segment comprises causing the pivoting segment to pivot at least 45 degrees relative to the main segment.

19. The method of claim 17, wherein causing the pivoting segment to pivot relative to the main segment comprises causing the pivoting segment to pivot at least 60 degrees relative to the main segment.

20. The method of claim 17, wherein causing the pivoting segment to pivot relative to the main segment comprises causing the pivoting segment to pivot at least 90 degrees relative to the main segment.
